(19) United States
(12) Patent Application Publication
(10) Pub. No.: US 2024/0423497 A1
(43) Pub. Date: Dec. 26, 2024

(54) A DEVICE AND METHOD FOR DETERMINING A RESPIRATORY SYSTEM INFECTION FROM EXHALED BREATH

(71) Applicant: PEXA AB, Göteborg (SE)

(72) Inventor: Svante HÖJER, Kode (SE)

(21) Appl. No.: 18/706,273

(22) PCT Filed: Nov. 2, 2022
(86) PCT No.: PCT/EP2022/080598

§ 371 Date: Apr. 30, 2024

(30) Foreign Application Priority Data
Nov. 2, 2021 (SE) 2151347-8

(51) Int. Cl. A61B 5/08; G01N 15/00; G01N 15/0205; G01N 33/497; G16H 10/60; G16H 50/70

(52) U.S. Cl. CPC A61B 5/082; G01N 15/0205; G01N 33/497; G16H 10/60; G16H 50/70; G01N 2015/0026

(57) ABSTRACT

A diagnostic device and method for detection of a respiratory system infection of a subject, comprising a particle detecting unit configured for obtaining data related to particles being exhaled from the subject's airways, a processing unit for determining the respiratory system infection based on the data from the particle detecting unit and subject related properties.

### BACKGROUND OF THE INVENTION

### Field of the Invention

This invention pertains in general to the field of detecting a respiratory system infection from exhaled breath. More particularly the invention relates to collecting particles from exhaled breath to detect a respiratory system infection based on variations in mass and/or size.

### Description of the Prior Art

Exhaled particles have been investigated to replay To reduce invasive diagnostics, such as bronchoalveolar lavage (BAL) and biopsies.

During these studies, it has been found that particles from exhaled breath may be used for continuously diagnosing and monitoring a subject connected to a respirator. This may be used for preventing structural damages. This has been described in, for example WO2013/117747 and in “Mechanically ventilated patients exhibit decreased particle flow in exhaled breath as compared to normal breathing patients”, Broberg, Ellen et al, ERJ Open Res 2020; 6: 00198-2019. Further studies has been published with respect to replacing Bal for mechanically ventilated subjects, for example “Increased particle flow rate from airways precedes clinical signs of ARDS in a porcine model of LPS-induced acute lung injury”, Stenlo, Martin et al, Am J Physiol Lung Cell Mol Physiol 318: L510-L517, 2020; and “Monitoring lung injury with particle flow rate in LPS- and COVID-19-induced ARDS”, Stenlo, Martin et al, Physiological Reports. 2021; 9:e14802. It has been found that different ventilation modes resulted in unique particle patterns and may be used as a fingerprint for the different ventilation modes, “Particle flow rate from the airways as fingerprint diagnostics in mechanical ventilation in the intensive care unit: a randomised controlled study”, Hallgren, Filip et al, ERJ Open Res 2021; 7: 00961-2020.

Lung functioning using particles has also been studied in relation to lung transplantation and lung cancer surgery. This is described in the thesis “Monitoring lung transplantation and lung cancer surgery using particles in exhaled air Preclinical and clinical implementation.” By Ellen Broberg, Doctoral Dissertation Series 2019:113; ISBN 978-91-7619-842-1.

Other areas that has been investigated is asthma, “Assessing small airways dysfunction in asthma, asthma remission and healthy controls using particles in exhaled air”, ERJ Open Res 2019; 5: 00202-2019.

Using exhaled breath for detecting a respiratory system infection has not been done.

In hospitals settings, there is a need for fast and reliable ways of detecting if a subject has a respiratory system infection. In particular, non-invasive methods that are not relying on sending samples to a laboratory would be an advantage.

Further advantageous would be improved cost-effectiveness compared to the invasive methods used today.

### SUMMARY OF THE INVENTION

Accordingly, embodiments of the present invention preferably seek to mitigate, alleviate or eliminate one or more deficiencies, disadvantages or issues in the art, such as the above-identified, singly or in any combination by providing a device, a system, and a method for detection of a respiratory system infection of a subject.

A first aspect of the disclosure relates to a diagnostic device for detection of a respiratory system infection of a subject. The device may include a particle detecting unit configured for obtaining data related to particles being exhaled from the subject's airways. The device may also include a processing unit for determining the respiratory system infection based on the data from the particle detecting unit and subject related properties.

In one example of the device, the particle detecting unit may be a particle counter or sizer, such as an optical based particle counter or sizer.

In one example of the device, the data may be any of number of particles, mass, size, mass distribution, size distribution.

In one example of the device, the particles may be aerosols derived from the subject's respiratory system.

In one example of the device, the data provides a pattern related to the respiratory system infection.

In one example of the device, the subject related properties may be information related to the respiratory system of the subject. The properties may include exhaled volume, number of exhalations, flow velocity of the exhaled breath, relative moist in the exhaled breath, temperature of the exhaled breath and/or oxygen saturation.

In one example of the device, the information related to the respiratory system of the subject may be information related to a physical condition and/or a health condition of the subject. The information may include weight, height, sex, age, medical records, smoker/non-smoker, and/or heart frequency.

In one example of the device, a reference database of healthy subjects may be compared with the measured data to determining the respiratory system infection. Such as, a reference database of healthy subjects may be compared with the measured data where subject related properties have been removed.

In one example of the device, a reference database of subjects with a diagnosed respiratory system infection may be compared with to determining the respiratory system infection. Such as, a reference database of subjects with a respiratory system infection may be compared with the measured data where subject related properties have been removed.

In one example of the device, the data may be filtered using the subject related properties before comparing the data with the reference database.

In one example of the device, the processing unit may be determining the respiratory system infections, qualitatively or quantitatively.

In one example of the device, the determination may be based on a pre-defined number of particles, such as a predetermined number of particles, or a predetermined total mass of counted particles.

In one example of the device, the data is collected during a predefined screening process.

In one example of the device, the predefined screening process may include at least one of, a pre-determined number of exhales, a predetermine inhalation and exhalation routine, exhalation after inhalation of pure air.

In one example of the device, thresholds may be used of to reduce the impact of too large particles and/or too small particles.

Another aspect of the disclosure relates to a method for detection of a respiratory system infection of a subject. The method may include receiving particle data from a particle detecting unit and wherein the particle data is related to particles being exhaled from the subject's airways. The method may further include determining the respiratory system infection based on the particle data from the particle detecting unit and subject related properties.

It should be emphasized that the term “comprises/comprising” when used in this specification is taken to specify the presence of stated features, integers, steps or components but does not preclude the presence or addition of one or more other features, integers, steps, components or groups thereof.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which

FIG. 1 is illustrating a schematic exemplary device for detection of a respiratory system infection;

FIG. 2 is a schematic flow-chart over an exemplary method for detection of a respiratory system infection;

FIGS. 3A and 3B are examples of measured data from a population with a respiratory system infection and a healthy population;

FIGS. 4A and 4B are examples of measured data from a population with a respiratory system infection and a healthy population; and

FIG. 5 is an example of differences between a particle size range of subjects with a respiratory system infection and healthy subjects.

### DESCRIPTION OF THE PREFERRED EMBODIMENTS

Specific examples for the disclosure will now be described with reference to the accompanying drawings. The disclosure may, however, be embodied in many different forms and should not be construed as limited to the examples set forth herein; rather, these examples are provided so that this disclosure will be thorough and complete, and will fully convey the scope of the disclosure to those skilled in the art. The terminology used in the detailed description of the examples illustrated in the accompanying drawings is not intended to be limiting of the disclosure. In the drawings, like numbers refer to like elements.

The following description focuses on an example of the present disclosure applicable to a device, system and method for detecting and/or diagnosing a respiratory system, such as an airway and/or lung, infection of a subject. A subject is could be a mammal, such as a human. The subject may be a patient. The detection and/or diagnosis is based on quantifying particles in exhaled air from the subject. For example, by detecting deviations from a normal state of the respiratory system, such as an airway or a lung, a respiratory system infection can be detected and/or diagnosed.

A respiratory system infection could be different type of virous and/or bacteria-based diseases affecting the respiratory system, such as upper respiratory infections, lower respiratory infections, cough, flue, covid-19, pneumonia, influenza, tuberculosis, inflammation, endothelial dysfunction, sepsis, septic shock etc.

Particles may herein be non-volatile particles, such as aerosols. The aerosols may be derived from said patient's airways. The particles are thought to be generated from surfaces of the airway mucus or respiratory tract lining fluid (RTLF) that covers the epithelial surface of the distal parts of the lung.

When parts a subject's respiratory system, e.g. the airways and/or lungs, gets infected it could affect the composition and structure of the surfactants and mucin in the respiratory system. These changes may alter droplet generation and droplet size during acts of breathing.

These changes may lead to the particle composition in the exhaled breath being affected. The changes to the particle composition may be seen in a change in the size distribution, mass distribution and/or number of exhaled particles compared to a healthy subject.

Other things that may cause changes to the composition and structure of the surfactants and mucins are physiological alterations of a subject's condition.

The present inventors have during their research found out that distribution of particles originating from the respiratory system and especially particles generated in the airways and lungs, may be used as a marker (like a fingerprint) to detect and/or diagnose infections.

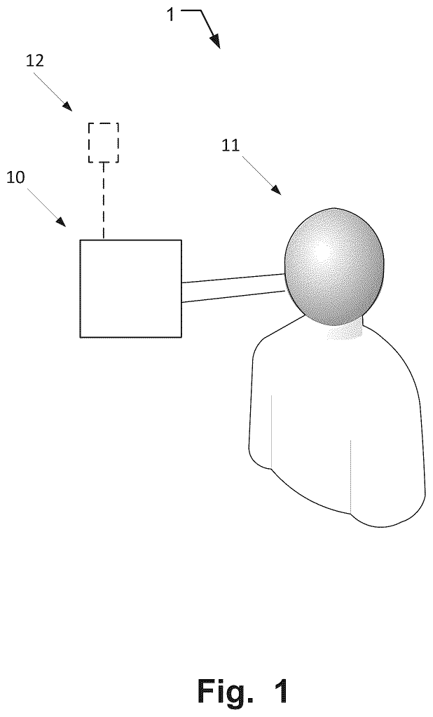

In FIG. 1, a schematic diagnostic device 1 for detection of a respiratory system infection of a subject 11 is illustrated. The device includes a particle detecting unit 10 into which the subject 11 may exhale.

The exhalation may be performed into a mouthpiece connected to a conduit. The conduit may further be connected to the particle detecting unit 10.

The particle detecting unit 10 may quantify the particles in real-time when the subject 11 exhales.

The particle detecting unit 10 may in some examples sort the particles according to their size or mass to obtain a distribution.

A particle distribution profile of the particles' distribution may be a measure of how many particles of a particular mass or size (or mass or size range) are present in the exhaled air.

The particle detection unit 10 may be, for example, a
particle counter such as a Grimm 1.108 optical particle counter (Grimm Aerosol Technik, Ainring, Germany), capable of counting, and sizing particles in size intervals from 0.3 to 20 micrometre. But other optical particle counters such as a Grimm 1.107 and 1.109 may be used.

Other manufacturers such as TSI have particle sizers but also time of flight equipment that may be used as particle detection units 10.

Other options may be, Non-optical, electrostatically, conductance, condensation particle counters, Quartz Crystal Microbalance (QCM), Surface Plasmon Resonance (SPR) or surface acoustic-wave (SAW) etc.

The particle detecting unit 10 may provide a number size distribution of the measured particles or a mass distribution, calculated from the measured number size distribution. In some examples of the particle detecting unit 10, particle-laden gas is passed through a small, well defined, intensely illuminated volume in a manner so that only one particle at a time is illuminated. The illuminated particle gives rise to a pulse of scattered light, the intensity of which is measured. Since the intensity of scattered light depends on the particle size, it is possible to count and size the particles in the air stream.

Time of flight may also be used as a measurement principle for a particle detecting unit 10. Here, the time of particle propagation from one laser beam to another is measured. The time it takes for the particle to move from one beam to the other depends on the particle's mass and/or size which may therefore be measured and characterised.

The device 1 may further include a processing unit 12. The processing unit is configured for determining a respiratory system infection based on the data obtained from the particle detecting unit 10. This may include the use of patient related properties to further improve the determination.

The processing unit 10 or data processing device may be implemented by special-purpose software (or firmware) run on one or more general-purpose or special-purpose computing devices. In this context, it is to be understood that each “element” or “means” of such a computing device refers to a conceptual equivalent of a method step; there is not always a one-to-one correspondence between elements/means and particular pieces of hardware or software routines. One piece of hardware sometimes comprises different means/elements. For example, a processing unit serves as one element/means when executing one instruction, but serves as another element/means when executing another instruction. In addition, one element/means may be implemented by one instruction in some cases, but by a plurality of instructions in some other cases. Such a software controlled computing device may include one or more processing units, e.g. a CPU (“Central Processing Unit”), a DSP (“Digital Signal Processor”), an ASIC (“Application-Specific Integrated Circuit”), discrete analog and/or digital components, or some other programmable logical device, such as an FPGA (“Field Programmable Gate Array”). The data processing unit 10 may further include a system memory and a system bus that couples various system components including the system memory to the processing unit. The system bus may be any of several types of bus structures including a memory bus or memory controller, a peripheral bus, and a local bus using any of a variety of bus architectures. The system memory may include computer storage media in the form of volatile and/or non-volatile memory such as read only memory (ROM), random access memory (RAM) and flash memory. The special purpose software may be stored in the system memory, or on other removable/non-removable volatile/non-volatile computer storage media which is included in or accessible to the computing device, such as magnetic media, optical media, flash memory cards, digital tape, solid state RAM, solid state ROM, etc. The data processing unit 10 may include one or more communication interfaces, such as a serial interface, a parallel interface, a USB interface, a wireless interface, a network adapter, etc., as well as one or more data acquisition devices, such as an A/D converter.

The special-purpose software may be provided to the control unit or data processing device on any suitable computer readable medium, including a record medium and a read-only memory.

There are various ways of determining an infection based on the characteristics of the data obtained by the particle detecting unit 10. The infection may be determined qualitatively or quantitatively. I.e. it is determined that the patient has an infection, or it is determined what type of infection the patent has. This may be determined by analysing the data. The data received from the particle detection unit 10 may be any of a number of particles, mass, size, mass distribution, and/or size distribution.

Analysing the size and/or mass distribution, the number of particles in a specific range, and/or total mass of particles in the exhaled breath and the distribution, the number of particles in a specific range and/or total mass may be different compared to a healthy person which may indicate the present of a respiratory system infection.

An infection may be determined by analysing the characteristics of the exhaled particles. For example, an infection may lead a shift in the mass and/or size distributions. The characteristics of a distribution from an infected patient has a tendency to shift towards larger or heavier particles compared to a healthy person. This means that an infection may qualitatively be determined by analysing the distribution of the particles, the number of particles in a specific range, or the total mass of the particles collected. This may be done by determining the total mass of a predefined number of exhaled particles or the number of particles in a specific range. A larger total mass compared to a healthy subject may therefore indicate the presence of an infection. Additionally, and/or alternatively, a larger number or particles in a specific size range compared to a healthy subject, may also indicate the presence of an infection.

Some infections may lead to a shift towards smaller and/or lighter particles. Analysing the size and/or mass distribution of the exhaled breath and the distribution is shifted towards more particles being smaller or lighter compared to exhaled breath of a healthy person may indicate that the subject has a respiratory system infection.

Similar, by looking at the total mass of the exhaled particles and the total mass is smaller compared to a healthy subject may indicate the presence of a respiratory system infection. Additionally, and/or alternatively, a smaller number or particles in a specific size range compared to a healthy subject, may also indicate the presence of an infection.

Further, some respiratory system infection may shift the mass and/or size distributions of particles both towards lighter/smaller and larger/heavier particles. Hence, analysing the distribution and the distribution is different from a healthy person may indicate the present of a respiratory system infection.

Further, the determination may be based on a pre-defined number of particles, such as a predetermined number of particles. Alternatively, and/or additionally, the determination may be based on a predetermined total mass of counted particles.

Different respiratory system infections may affect the characteristics of the exhaled particles differently, e.g. different respiratory system infections may have different size and/or mass distribution of exhaled particles. By analysing the characteristic of the size and/or mass distribution of exhaled particles, a respiratory system infection may be determined quantitively, i.e. not only the presence of a respiratory system infection may be determined by also the type of respiratory system infection may be established. The characteristics of the size and/or mass distribution may be a pattern related to a respiratory system infection. The patterns may function as a fingerprint of a respiratory system infection.

By comparing the characteristics and/or pattern of the size and/or mass distribution of exhaled breath with a reference database of healthy subjects, it may be determined that a patient has a respiratory system infection.

The characteristics and/or pattern of the size and/or mass distribution of exhaled breath may be compared with a reference database of subjects with respiratory system infections to establish the respiratory system infection, such as the type of respiratory system infection.

It has been found that the characteristics of the size and/or mass distribution, as well as the total weight or the number of particles in a specific range, of exhaled particles may vary between individuals due to individual properties. These individual properties may include information related to the airways of the subject. The information related to the airways of the subject may include, exhaled volume, number of exhalations, flow velocity of the exhaled breath, relative moist in the exhaled breath, temperature of the exhaled breath and/or oxygen saturation. Most of this is data that may be measured and used when obtaining the size and/or mass distribution of exhaled particles, total weight and/or the number of particles in a specific range. The device may therefore include a further device for measuring these individual properties.

By taking such properties into account, individual variations may be removed from the mass and/or size distribution, which may improve the determination of a respiratory system infection. This may especially improve the accuracy when using the size and/or mass distribution to establish the type of respiratory system infection a subject may have.

One way of removing the variation due to individual properties, may be to normalize the data based on the individual properties. Another way is to filter the data based on the individual properties. For example, by comparing exhaled breath data with exhaled breath data of subjects with similar individual properties.

As part of the method of determining a respiratory system infection of a subject, individual properties may first be established.

Additionally, and/or alternatively, the information related to the respiratory system of the subject may be information related to a physical condition and/or a health condition of said subject. This information may include weight, height, sex, age, medical records, smoker/non-smoker, and/or heart frequency characteristics. Information that may cause variation in the size and/or mass distribution not related to a respiratory system infection.

As previously described, this information may be used to normalize the data or filtering the data to improve the detection of an infection by removing variations in the size and/or mass distribution that is not related to variations caused by a respiratory system infection.

To further improve the detection of a respiratory system infection the collection of data from the exhaled breath may be conducted during a predefined screening process. A predefined screening process may aid in standardizing the data and cause less variations in the collected distributions not caused by a respiratory system infection. Less variation in the collected data, not caused by an air infection, may provide an improvement when it comes to comparing the collected data with data in a reference database.

The device may include means, such as a screen, to prompt the user in how to perform a predefined screening process. This may be done by either text or by illustrations visualizing the steps to be perform during the predefined screening process.

The device may include different predefined screening processes to be selected from.

A predefined screening process may include at least one of, a pre-determined number of exhales, a predetermine inhalation and exhalation routine, exhalation after inhalation of pure air.

The device for detecting a respiratory system infection may also include means for performing the screening process. For example, the device may include pure air connected to the same mouthpiece as the exhalation is performed through.

The data may also be collected by having the subject to exhale for a predefined period of time, and/or a predefined number of times.

Additionally, and/or alternatively, the collected data may also be standardized by using thresholds for the particles. For example, threshold may be used for reducing the impact of particles having a size and/or mass that is not within an expected range used for detecting respiratory system infection. The threshold may be used to remove data that relates to particles considered to be too large and/or too small. In some examples, the threshold may be used to only collect particles of a specific size range known to have a large variation when comparing healthy subjects to infected subjects. The total mass of a particle range may be used and compared with the total mass of the same group of known healthy subjects to determine if a subject is infected or not. Additionally, and/or alternatively, the number of particles in a specific range may be used and compared with the number of particles in the same specific range of the same group of known healthy subjects to determine if a subject is infected or not

Further, in some example, the threshold may be relevant when using total mass of exhaled particles to detect a respiratory system infection. When collecting a predefined number of particles for the total mass, particles that are too large or too small (outliers) may have an impact on the data that may provide positive negatives or negative positives.

The device may be used for monitoring, such as continuously monitoring, the development of a respiratory system infection by analysing variations in the exhaled particles. This may be used to determine if a subject is getting sicker or if subject's health is improving. This may be used to check the impact of a medication on a subject as well.

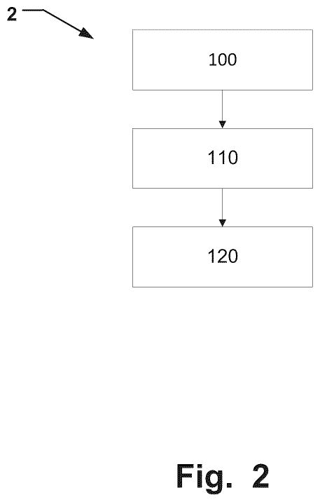

FIG. 2 is illustrating a schematic flow-chart 2 over an exemplary method for detection of a respiratory system infection of a subject. The method may be a computer implemented method. The computer implement method may be implements as a computer software to be executed on a computer or processor and having code for implementing the method steps. The software may be part of a computer of the device described above or be running on an external machine, such as in the cloud. The detection device may communicate with such external machine via known protocols.

The method 2 may comprise receiving 100 particle data from a particle detecting unit. Alternatively, the method may include obtaining particle data using a particle detecting unit. The particle data is related to particles being exhaled from the subject's airways.

The method may then include entering 110 subject related properties. The subject related properties may be entered using an input device connected to a processing unit of the detection device. The input device may be a keyboard or a touch screen. Additionally, and/or alternatively, the subject related properties may be entered by measuring the properties using measuring means/devices connected to the device, such as a scale, a spirometer, speed of exhaled air and/or volume. Some of these properties may be measured at the same time as the exhaled particle data is collected,

The method 2 may further include determining 120 the respiratory system infection based on the particle data received from, or obtained by, the particle detecting unit and the subject related properties.

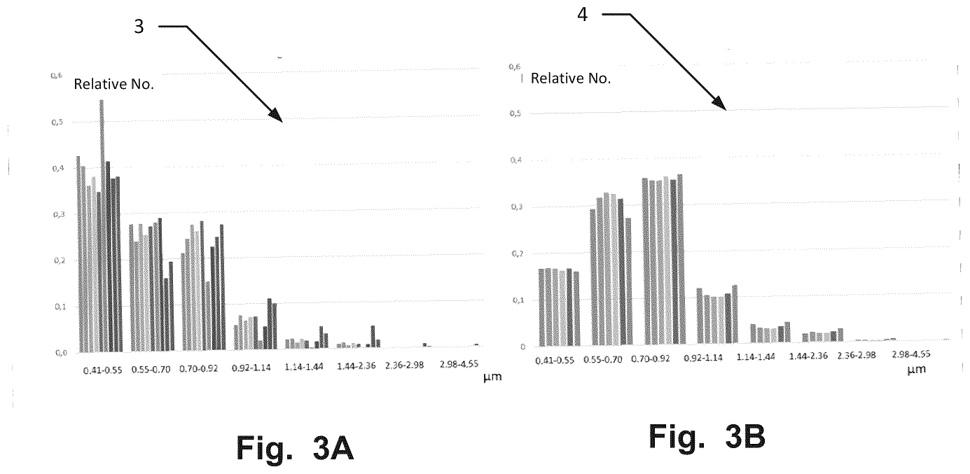

FIG. 3A is measured particle data 3 from subjects having pneumonia. The data is presented as a relative number distribution of exhaled particles in nine individuals with pneumonia. FIG. 3B is measured particle data 4 from healthy subject. The data is presented as a relative number distribution of exhaled particles in six healthy subjects.

From the data it is clear that there is a change in the distribution between healthy subjects and infected subjects. The distribution over infected subjects can be seen as a finger print for pneumonia and may be used for determining that a subject has pneumonia using any of the methods described above.

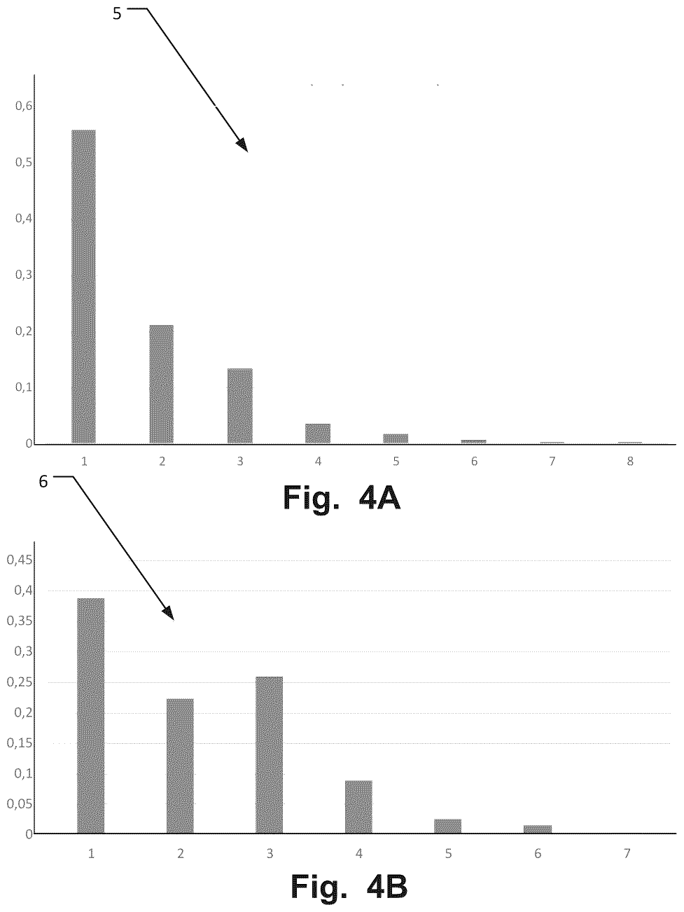

FIGS. 4A and 4B are measured particle data from subjects having Covid-19 6 and from healthy subjects 5. The data illustrates the size distribution of the number of particles (median) measured from 10 subjects diagnosed with Covid-19 and 100 healthy subjects. Each number on the X-axis represents a bin. Each bin has been assigned a particle range. Again, from the data it is clear that there is a change in the distribution between healthy subjects and infected subjects. The distribution over infected subjects can be seen as a fingerprint for Covid-19 and may be used for determining that a subject has Covid-19 using any of the methods described above.

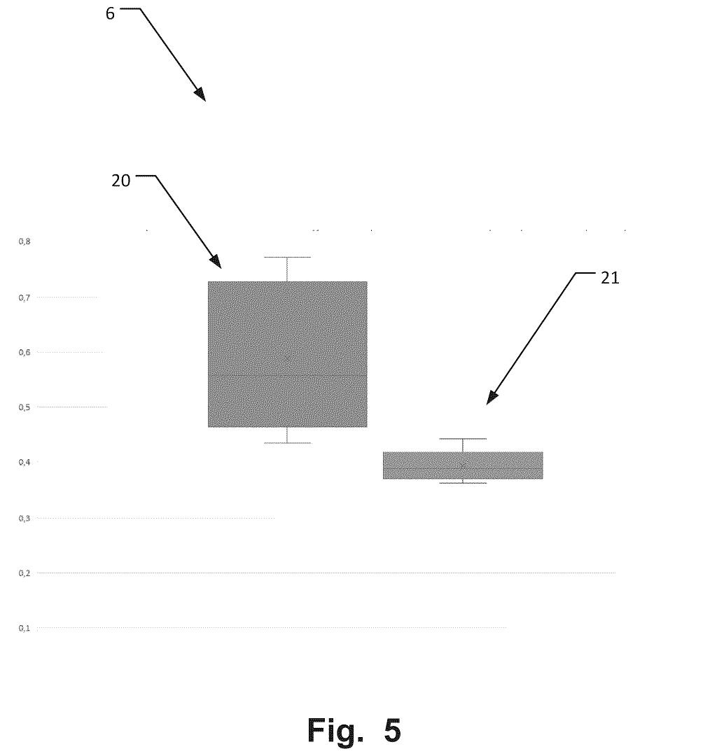

FIG. 5 is illustrating the ratio of exhaled particles in the range <0.41-0.55 mua between subjects having Covid-19 20 and healthy subjects 21. Again, a fairly large difference can be detected between healthy subjects and infected subjects. This difference may be used to determine that a subject has an infection or not.

Embodiments of the present invention are described herein with reference to flowchart and/or block diagrams. It will be understood that some or all of the illustrated blocks may be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

It is to be understood that the functions/acts noted in the diagrams may occur out of the order noted in the operational illustrations. For example, two blocks shown in succession may in fact be executed substantially concurrently or the blocks may sometimes be executed in the reverse order, depending upon the functionality/acts involved. Although some of the diagrams include arrows on communication paths to show a primary direction of communication, it is to be understood that communication may occur in the opposite direction to the depicted arrows.

The present invention has been described above with reference to specific embodiments. However, other embodiments than the above described are equally possible within the scope of the invention. Different method steps than those described above, performing the method by hardware or software, may be provided within the scope of the invention. The different features and steps of the invention may be combined in other combinations than those described. The scope of the invention is only limited by the appended patent claims.

1. A diagnostic device for detection of a respiratory system infection of a subject, comprising:
a particle detecting unit configured for obtaining data related to particles being exhaled from said subject's airways;
a processing unit for determining said respiratory system infection based on said data from said particle detecting unit and subject related properties.

2. The diagnostic device of claim 1, wherein said particle detecting unit is a particle counter or sizer, such as an optical based particle counter or sizer.

3. The diagnostic device of claim 1, wherein said data is any of number of particles, mass, size, mass distribution, size distribution.

4. The diagnostic device of claim 1, wherein said particles are aerosols derived from said subject's respiratory system.

5. The diagnostic device of claim 1, wherein said data provides a pattern related to said respiratory system infection

6. The diagnostic device of claim 1, wherein said subject related properties are information related to the respiratory system of said subject, including, exhaled volume, number of exhalations, flow velocity of the exhaled breath, relative moist in the exhaled breath, temperature of the exhaled breath and/or oxygen saturation.

7. The diagnostic device of claim 6, wherein said information related to the respiratory system of said subject are information related to a physical condition and/or a health condition of said subject, including weight, height, sex, age, medical records, smoker/non-smoker, or heart frequency.

8. The diagnostic device of claim 1, wherein a reference database of healthy subjects is compared with to determining said respiratory system infection.

9. The diagnostic device of claim 1, wherein a reference database of subjects with a respiratory system infection is compared with to determining said respiratory system infection.

10. The diagnostic device of claim 8, wherein said data is filtered using said subject related properties before comparing said data with said reference database.

11. The diagnostic device of claim 1, wherein said processing unit is determining said respiratory system infections, qualitatively or quantitatively.

12. The diagnostic device of claim 1, wherein said determination is based on a pre-defined number of particles, such as a predetermined number of particles, or a predetermined total mass of counted particles.

13. The diagnostic device of claim 1, wherein said data is collected during a predefined screening process.

14. The diagnostic device of claim 13, wherein the predefined screening process includes at least one of, a pre-determined number of exhales, a predetermine inhalation and exhalation routine, exhalation after inhalation of pure air.

15. The diagnostic device of claim 1, wherein thresholds are used of to reduce the impact of too large particles and/or too small particles.

16. A computer implemented method for detection of a respiratory system infection of a subject, comprising:
receiving particle data from a particle detecting unit and wherein said particle data is related to particles being exhaled from said subject's airways;
determining said respiratory system infection based on said particle data from said particle detecting unit and subject related properties.

17. A computer program comprising instructions which, when the program is executed by a computer, cause the computer to carry out the method of claim 16.
